(19) United States
(12) Patent Application Publication
(10) Pub. No.: US 2022/0170904 A1
(43) Pub. Date: Jun. 2, 2022

(54) SYSTEMS AND METHODS FOR MONITORING BIOLOGICAL TISSUE DEGRADATION

(71) Applicant: Arizona Board of Regents on behalf of Arizona State University, Scottsdale, AZ (US)

(72) Inventors: Abigail Hanson, Tempe, AZ (US); Tyler Grenier, Gilbert, AZ (US); Nandini Sharma, Scottsdale, AZ (US); Claudia Fragoso, Tempe, AZ (US); Carolyn Compton, Scottsdale, AZ (US); George Moakley, Phoenix, AZ (US)

(73) Assignee: Arizona Board of Regents on behalf of Arizona State University, Scottsdale, AZ (US)

(21) Appl. No.: 17/539,385

(22) Filed: Dec. 1, 2021

Related U.S. Application Data
(60) Provisional application No. 63/119,849, filed on Dec. 1, 2020.

(51) Int. Cl. G01N 33/483; G08B 5/36; G01N 33/00

(52) U.S. Cl. CPC G01N 33/4833; G01N 33/0047; G08B 5/36

(57) ABSTRACT

Disclosed herein are systems and methods for monitoring biological tissue degradation. A container comprises a body having an interior compartment, a lid having an interior surface and a sensing unit configured to obtain data about an environmental condition, the sensing unit in electrical communication with a processing unit. The lid of the container, when coupled to the body, encloses the sensing unit within the interior compartment. The processing unit comprises components in electrical communication, including a processor, a non-transitory processor-readable storage medium, a power source, and an indicator. The storage medium contains instructions that cause the processor to process and store the data, and determine a status, for which the indicator may be configured to display an indication. A method comprises obtaining the container, placing the tissue in the interior compartment, coupling the lid to the body, activating the sensing unit and the processing unit, and observing the indicator.

### CROSS-REFERENCE TO RELATED APPLICATIONS

This application claims priority to and benefit of U.S. Provisional Application Ser. No. 63/119,849, filed Dec. 1, 2020, entitled “Systems and Methods for Monitoring Biological Tissue Degradation,” which is incorporated herein by reference in its entirety.

### TECHNICAL FIELD

The present disclosure relates generally to systems and methods for monitoring the degradation of biological tissue. The systems and methods described herein may be used in a variety of applications, including to monitor the quality of and degradation of biopsy samples, excised tissues, and the like.

### BACKGROUND

A biopsy is a common medical procedure performed to remove one or more small samples of tissue from a patient for subsequent laboratory testing. When tissue is excised from a patient, the cells are removed from their native blood supply and temperature-controlled environment. This induces significant biological stress that alters metabolic processes within the tissue. As a result, the molecular composition and integrity of the excised tissue undergo significant changes, which may result in artifactual results from subsequent molecular analysis, leading to misdiagnosis. In some instances, as a result of metabolomic shifts, the excised tissue may emit volatile organic compounds (VOCs) in levels that correspond to or can be correlated with the degradation of the tissue itself. Therefore, there exists a need for a quantitative method for tracking the effects of these stresses on excised tissue samples to determine whether the quality of the tissue samples has deviated from or deteriorated relative to the quality of the tissue before it was excised.

### SUMMARY

The instant disclosure is directed to systems and methods for monitoring metabolic stress and tissue degradation in biological specimens. In an embodiment, a container may comprise a body having an interior compartment, a lid having an interior surface and a sensing unit affixed to the interior surface, and a processing unit in electrical communication with the sensing unit. The lid of the container may be configured to be coupled to the body of the container, thereby enclosing the sensing unit within the interior compartment of the body. The sensing unit may further be configured to obtain data about an environmental condition such as an environmental condition within the interior compartment of the body.

In an embodiment, the processing unit of the container may comprise a processor, a non-transitory, processor-readable storage medium in electrical communication with the processor, a power source in electrical communication with the processor and the processor-readable storage medium, and an indicator in electrical communication with the processor, the processor-readable storage medium, and the power source. The processor-readable storage medium may contain instructions that, when executed, cause the processor to process and store the data about the environmental condition, and determine a status based on the data. The indicator may be configured to display an indication corresponding to the status. In certain embodiments, the sensing unit may be configured to detect volatile organic compounds (VOCs), and the environmental condition may include a presence of VOCs. In some embodiments, the container may be a sterile biopsy container.

In an embodiment, a method for monitoring degradation of a biological tissue may comprise obtaining a container as described herein, placing the biological tissue in the interior compartment of the body, and coupling the lid to the body. The method may further comprise activating the sensing unit and the processing unit, thereby executing the instructions and determining the status, and observing the indicator, thereby monitoring the degradation of the biological tissue.

### BRIEF DESCRIPTION OF THE DRAWINGS

FIG. 1 is a schematic representation of a conventional biopsy container.

FIG. 2A is a schematic representation of an embodiment of a container comprising a body, a lid, a sensing unit, and a processing unit, in accordance with the present disclosure.

FIG. 2B is a schematic representation of an alternative view of the lid of the container shown in FIG. 2A, in accordance with the present disclosure.

FIG. 3A shows an alternative embodiment of a container comprising a body, a lid, a sensing unit, and a processing unit, in accordance with the present disclosure.

FIG. 3B shows an alternative view of the lid of the container shown in FIG. 3A, in accordance with the present disclosure.

FIG. 4 is a first graph showing the total concentration of volatile organic compounds (VOCs, in ppb) as a function of time (in seconds), in accordance with the present disclosure.

FIG. 5 is a second graph showing the total concentration of volatile organic compounds (VOCs, in ppb) as a function of time (in seconds), in accordance with the present disclosure.

FIG. 6 is a graph showing the percent area of 3,4-Dihydroisoquinolin-7-ol, 1-[4-hydroxybenzyl]-6-methoxy- in tissue headspace samples at designated pro-degradation condition time points, in accordance with the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

All percentages, parts and ratios are based upon the total weight of the compositions and all measurements made are at about 25° C., unless otherwise specified.

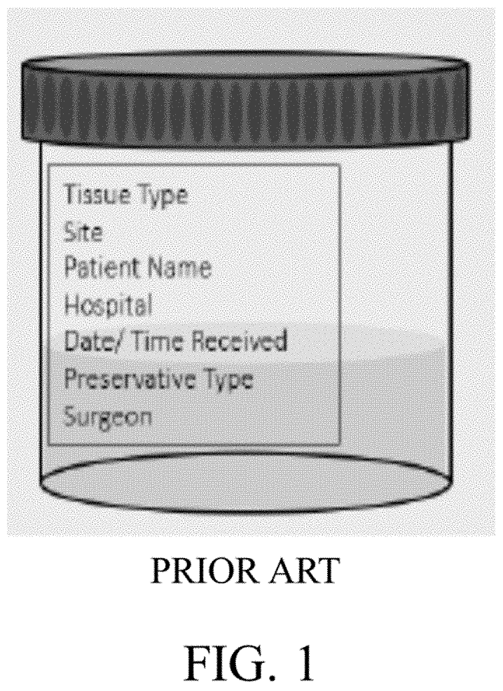

A biopsy is a common medical procedure in which a sample of biological tissue is removed from a patient for subsequent laboratory analysis and testing. A biopsy may be conducted, for example, when a healthcare provider suspects cancer, an inflammatory condition, or an autoimmune disorder. Approximately 3 million biopsies are performed annually in the United States. The preservation and tracking of biopsied tissue samples' molecular quality presents a challenge. When tissue is excised from a patient, the cells are removed from their native blood supply and temperature-controlled environment, thereby causing significant biological stress to the excised tissue. FIG. 1 is a schematic representation of a conventional biopsy container. Standard processes do not provide a way to track the effects of these stresses on the excised tissue to determine whether the biology of the excised tissue has deviated from the biology of the patient's intact tissues. Without a way to track these effects, pathologists and other analysts must trust that the tissue sample they receive is of high quality and fit for its intended analytical purpose. Similarly, healthcare professionals must trust that the data resulting from the analysis of the excised tissue(s) is accurate for diagnosis and treatment purposes. Compromised tissue samples may thus not accurately reflect the biology of a disease, even when the patient actually has the disease. Providing for systems and methods for monitoring the degradation of these excised biological tissues may allow pathologists and healthcare providers to more accurately determine the status of a tissue sample and thus more accurately analyze a patient's health.

Volatile organic compounds (VOCs) are released from tissues as they degrade. VOCs are carbon-based chemicals emitted from the human body (as well as other sources), and they can reflect a person's metabolic condition. There are more than 1,300 known VOCs within humans, and a number of these VOCs exist in every healthy tissue sample. However, when a tissue is diseased, the VOCs that the tissue emits change compared to a healthy tissue. The compounds emitted from a diseased tissue may provide some indication of the disease from which the tissue is suffering. Without wishing to be bound by theory, there may be a relationship between the change in concentration of VOCs emitted by a tissue sample and the molecular quality or health of that tissue sample. Such a relationship may indicate the quality of the excised tissue as compared to the site of a patient's body from which the tissue was excised. Other changes in environmental conditions, including changes in temperature, humidity, oxygen saturation, pH, and the like, may also indicate the quality of an excised tissue sample as compared to the site of the patient's body from which the tissue was excised. Environmental changes, including the emission of VOCs, may be monitored, for example, by one or more sensors. Such sensors are conventionally used to monitor VOC emission for purposes of assessing indoor air quality, contents of exhaled or emitted air, and the like. Systems and methods for monitoring biological tissue degradation, such as the degradation that an excised tissue sample may undergo, are described herein.

In an embodiment, a container may comprise a body and a lid. The body of the container may have an interior compartment, and the lid of the container may have an interior surface. In an embodiment, the lid may be configured to be coupled to the body of the container. The lid may be configured to be, for example, removably coupled to the body of the container, partially coupled to the body of the container, fixedly coupled to the body of the container, and the like.

In some embodiments, the lid may further comprise a sensing unit. In an embodiment, the sensing unit may be affixed to the interior surface. The sensing unit may be, for example, removably affixed to the interior surface, or it may be fixedly attached (e.g., adhered) to the interior surface. In an embodiment, the sensing unit may be configured on or affixed to the lid such that, when the lid is coupled to the body of the container, the sensing unit is enclosed within the interior compartment of the body of the container.

In some embodiments, the sensing unit may comprise one or more devices selected from the group consisting of an electrochemical sensor, a gas sensor, a metal oxide sensor, a gas chromatography apparatus, a mass spectrometry apparatus, a photo-ionization detector, equivalents thereof, and combinations thereof. In certain embodiments, the sensing unit may be configured to detect VOCs. In an embodiment, the sensing unit may comprise a metal oxide sensor configured to detect VOCs.

In an embodiment, the sensing unit may be configured to obtain data about one or more environmental conditions. In an embodiment, the one or more environmental conditions may comprise one or more ambient environmental conditions. In some embodiments, the environmental condition may be, for example, a temperature, a presence of VOCs, a humidity level, an oxygen saturation level, a pH, or any combination thereof. In an embodiment, the environmental condition may comprise a temperature ranging from about 18 degrees Celsius to about 30 degrees Celsius. The temperature may be, for example, about 18 degrees Celsius, about 19 degrees Celsius, about 20 degrees Celsius, about 21 degrees Celsius, about 22 degrees Celsius, about 23 degrees Celsius, about 24 degrees Celsius, about 25 degrees Celsius, about 26 degrees Celsius, about 27 degrees Celsius, about 28 degrees Celsius, about 29 degrees Celsius, about 30 degrees Celsius, or any range between any two of these values, including endpoints.

In an embodiment, the environmental condition may be, for example, a presence of VOCs. The presence of VOCs may be measured in parts per billion (ppb). In some embodiments, the presence of VOCs may be from about 1 ppb to about 60,000 ppb. The presence of VOCs may be, for example, about 1 ppb, about 500 ppb, about 1,000 ppb, about 2,000 ppb, about 5,000 ppb, about 10,000 ppb, about 15,000 ppb, about 20,000 ppb, about 25,000 ppb, about 30,000 ppb, about 35,000 ppb, about 40,000 ppb, about 45,000 ppb, about 50,000 ppb, about 55,000 ppb, about 60,000 ppb, or any range between any two of these values, including endpoints. In an embodiment, the environmental condition may be measured with a sensitivity of about 10 ppb. In some embodiments, the environmental condition may be measured with an accuracy of about 1 ppb. Table 1 below lists non-limiting examples of VOCs that may be detected. In some embodiments, for example, the VOCs as described herein may comprise one or more VOCs listed in Table 1, derivatives thereof, or combinations thereof.

TABLE 1
(CH3)2NCl
3,4-Dimethoxycinnamic acid
3-Methoxybenzoic acid, 3-pentyl ester
3,4-Dihydroisoquinolin-7-ol, 1-[4-hydroxybenzyl]-6-methoxy-
N-[3-Methylaminopropyl]aziridine
2,3-Dimethylquinizarin, 2-methylpropionate
5,6,6,7-Tetramethyl-13-oxa-5,7-diazatetracyclo
[6.6.2.0(4,16).0(11,15)]hexadeca-1,3,8,10,15-pentaen-12-one
1H-indole, 3,3′-[1,4-phenylenedi-2,1-ethenediyl]bis[1,2-dimethyl-
5-(4-Methoxy-phenyl)-[1,3,4]oxadiazole-2-thiol
Cobalt, hexamethylbenzene-pentamethylcyclopentadienyl-
4H-1-Benzopyran-4-one, 6,7-dimethoxy-3-phenyl-
4,6-Heptadiyn-3-one
1,2,4-Benzenetricarboxylic acid, 1,2-dimethyl ester
Dimethyl 2-(3,4-dimethoxyphenyl)malonate
.beta.-Phenylpropiophenone
9H-fluorene-2,7-dicarboxylic acid, 9-oxo-
Heptafluorobutyramide, N-(2-butyl)-N-methyl-
Ethyl [5-hydroxy-1-(6-methoxy-4-methyl-3-quinolinyl)-3-
methyl-1H-pyrazol-4-yl]acetate
Propane, 2-chloro-2-nitro-
Anthranilic acid, N-methyl-, butyl ester
Morpholine, 4-(4-nitrophenyl)-
3-Phenpropenoic acid, 3-methoxy-6-(3,4-dimethoxyphenyl)-,
ethyl ester
Propanedinitrile, 2-(5-phenylthio-2-thienylmethylene)-
Benzene, [3-(1-methylethoxy)-5-hexenyl]-
Phenylacetic acid, 2-(1-adamantyl)ethyl ester
N,N-Dimethylnicotinamide
Phthalic acid, octyl tridec-2-yn-1-yl ester
Tricyclo[3.2.1.0(2,4)]octane, 8-methylene-,
(1.alpha.,2.alpha.,4.alpha.,5.alpha.)-
Butyrophenone, 2-phenyl-
Ketoprofen methyl ester
4,4′-bi-4H-pyran, 2,2′,6,6′-tetrakis(1,1-dimethylethyl)-4,4′-dimethyl-
Ethylamine, N,N-dinonyl-2-(2-thiophenyl)-
1-Pentene, 4,4-dimethyl-
Ethylamine, N,N-dioctyl-2-(2-thiophenyl)-
Pyrazolo[1,5-a]pyridine, 3-methyl-2-phenyl-
1,2-Benzenedicarboxylic acid, 4-methyl-, dimethyl ester
Tricyclo[5.2.1.0(1,5)]dec-5-en-8-ol
Acridine, 9,10-dihydro-9,9,10-trimethyl-
[1,1′-Biphenyl]-4-ol, 3,5-bis(1,1-dimethylethyl)-
Ethylamine, N,N-dinonyl-2-phenylthio
1,2,3-Triphenyl-3-methyl-cyclopropene
Benzofuran-5,6-diol-3-one, 2-benzylidene-
Coumarin, 6-benzyloxy-3,4-dihydro-4,4-dimethyl-5-nitro-
Acetamide, N-[3-hydroxy-4-(1-oxopropyl)phenyl]-
Hydrazinecarboxylic acid, 2-benzoyl-, ethyl ester
Ethyl homopiperonylate
2-(p-Methoxyanilino)-N,N-dimethylacetamide
Glutaric acid, di(3-methyl-2-nitrobenzyl) ester
3-Phenylpropionic acid, 3-pentyl ester
2-Butylamine, N-hexadecyl-
6H-Benzofuro[3,2-c][1]benzopyran, 3,9-dimethoxy-
2-oxo-4-phenyl-6-(4-chlorophenyl)-1,2-dihydropyrimidine
3-Trifluoromethylbenzyl amine, N,N-diundecyl
Dezaguanine
3-Phenylpropionic acid, 2-dimethylaminoethyl ester
Benzaldehyde, 2-nitro-, phenylhydrazone
Ethylbenzene
Benzene, 1,3-dimethyl-
1-Phenyl-5-methylheptane
2-Propyl-1-pentanol
5-Methyl-3-phenyl-1H-indazole
1-Triazene, 3,3-dimethyl-1-phenyl-
9,10-Anthracenedione, 1,4-dihydroxy-2,3-dimethyl-
7H-Dibenzo[b,g]carbazole, 7a,8-dihydro-7a-methyl-
2,4-Di(2,2,6,6-tetramethyl-1,2,5,6-tetrahydro-4-pyridyl)pyrrole
Phthalic acid, di(2,3-dimethylphenyl) ester
Ethanediamide, N,N′-bis(phenylmethyl)-
2-Butanamine, 3-methyl-
Acetamide, 2-fluoro-
D-Alanine, N-(4-anisoyl)-, ethyl ester
(4-Methylbenzyl)(4-morpholin-4-ylphenyl)amine
Perhydrohistrionicotoxin, 2-depentyl-, methoxyformate(ester)
1-Methylene-2-benzyloxy-cyclopropane
Pyridine, 3-ethyl-
Nicotinic acid, 4-nitrophenyl ester
4-p-Tolylcarbamoyl-butyric acid
4-(2-Hydroxyethyl)-2-phenyl-1,3-dioxane
3H-1,2,4-Triazole-3-thione, 2,4-dihydro-4-phenyl-5-(phenylamino)-
1-Methyl-2-phenylbenzimidazole
[3-(4-Methoxyphenyl)-4,5-dihydro-1,2-oxazol-5-yl]methanol
Methyl 5-acetyl-2-methoxybenzoate
1,2,4-Thiadiazol-5-amine, 3-phenyl-
1,4-Benzenedicarboxylic acid, 2-amino-, dimethyl ester
4-Benzyloxy-2-methoxymethoxy-phenol
2-Propenoic acid, 3-[4-(acetyloxy)-3-methoxyphenyl]-, methyl ester
3,4-Methylenedioxy-N,N-diethylbenzylamine
Dimethylsulfoxonium formylmethylide
Benzene 1,1′-[1,2-ethanediylbis(oxymethylene)]bis-
Dibenz[a,c]cycloheptan-9-amine, 2,3,4-trimethoxy-N-acetyl-
2,3,4,6-Tetrahydroisoquinolin-6-one, 1-[4-hydroxybenzyl]-
7-methoxy-
Acetic acid, 7,7,10a,12a-tetramethyl-2,5-dioxo-
1,2,3,4,4a,4b,5,7,8,9,10,10a,10b,11,12,12a-
hexadecahydro-1-azachrysen-8-yl ester
Butylphosphonic acid, neopentyl propyl ester
1,3-Diazetidine, 2,4-bis(hexafluoroisopropylideno)-1,3-diphenyl-

In some embodiments, the container may further comprise a processing unit. In an embodiment, the processing unit may be in electrical communication with the sensing unit, as described herein. In some embodiments, the processing unit may comprise at least one of: (i) a processor, (ii) a non-transitory, processor-readable storage medium, (iii) a power source, and (iv) an indicator. Each of these components may be in electrical communication with one another. For example, the non-transitory, processor-readable storage medium may be in electrical communication with the processor. The power source may be in electrical communication with the processor and the processor-readable storage medium. The indicator may be in electrical communication with the processor, the power source, and optionally, the processor-readable storage medium.

In certain embodiments, the processor-readable storage medium may contain instructions that, when executed, cause the processor to process and store the data about the one or more environmental conditions obtained by the sensing unit. In some embodiments, the processor-readable storage medium may be selected from the group consisting of a secure digital (SD) card, a receiver wirelessly coupled to an external server, and combinations thereof. In an embodiment, the power source may comprise one or more batteries.

In some embodiments, the processing unit may be configured to be activated and/or deactivated when the configuration of the container is changed. In an embodiment, the processing unit may be configured to activate when the lid contacts the body. In another embodiment, the processing unit may be configured to activate when the lid is coupled to the body, as described herein. In certain embodiments, the processing unit may be configured to deactivate when the lid is uncoupled from the body. In some embodiments, the processing unit may be configured to record a time of activation, a time of use, a duration of use, or any combination thereof.

In some embodiments, the processor-readable storage medium may also contain instructions that, when executed, cause the processor to determine a status based on the data about the one or more environmental conditions obtained by the sensing unit. In an embodiment, the sensing unit may comprise a metal oxide sensor configured to detect VOCs, and the status may be based on a presence of VOCs. In certain embodiments, the processing unit may be configured to determine the status by comparing the data from the sensing unit to one or more predetermined thresholds. In some embodiments, the status may be, for example, “viable,” “mildly degraded,” “fully degraded,” any intermediates thereof, or any combination thereof.

In certain embodiments, the indicator may be configured to display an indication corresponding to the status determined by the processor, as described herein. In some embodiments, the indicator may comprise a display. In certain embodiments, the indicator may comprise at least one light emitting diode (LED). In some embodiments, the indicator may be configured to display one or more indications that correspond to the status determined by the processor. In an embodiment, the one or more indications may include, for example, a first color when the status is “viable,” a second color when the status is “mildly degraded,” a third color when the status is “fully degraded,” or any combination thereof. In an embodiment, each color may be any conventional color; for example, the first color may be green, the second color may be yellow, the third color may be red, or any other color scheme.

In some embodiments, the interior compartment of the body of the container may be configured to house one or more biological tissues or excised tissue samples. In certain embodiments, the container may be a portable container. In an embodiment, the container may be a sterile biopsy container. In some embodiments, the container may be a conventional biopsy container. In certain embodiments, the container may be a portable sterile biopsy container.

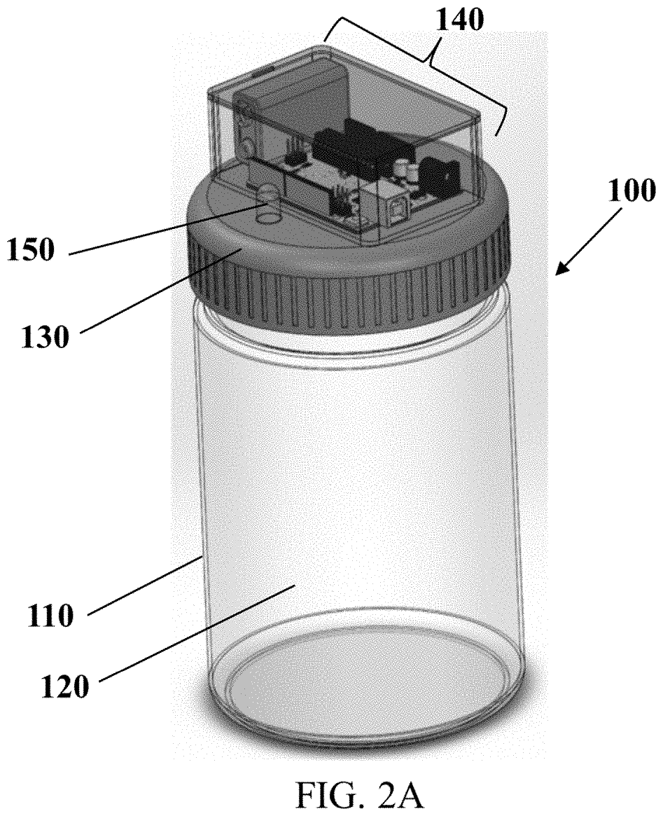

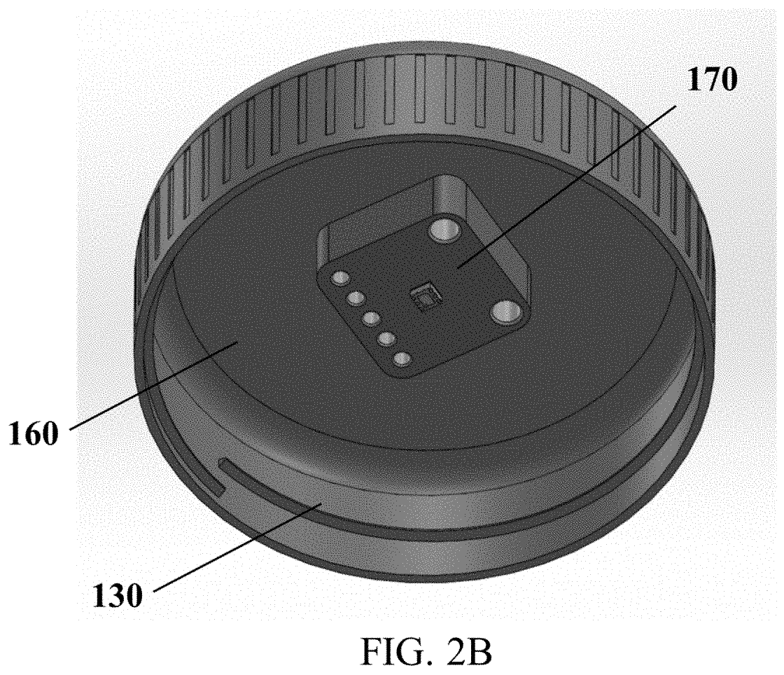

FIG. 2A and FIG. 2B show alternative views of an embodiment of a container 100 as described herein. FIG. 2A is a schematic representation of an embodiment of a container 100 comprising a body 110 having an interior compartment 120, a lid 130, a sensing unit (not shown), a processing unit 140, and an indicator 150. In this embodiment, a portion of the lid 130 and a portion of the body 110 are threaded, such that the lid 130 is configured to be removably coupled to the body 110. The indicator 150 comprises an LED, as described herein. FIG. 2B is a schematic representation of an alternative view of the lid 130 of the container 100 shown in FIG. 2A. The lid 130 of FIG. 2B has an interior surface 160, and a sensing unit 170 affixed to the interior surface 160 of the lid 130. The sensing unit 170 is affixed to the interior surface 160 of the lid 130 such that when the lid 130 and the body 110 are removably coupled, the sensing unit 170 is enclosed within the interior compartment 120 of the body 110 of the container 100.

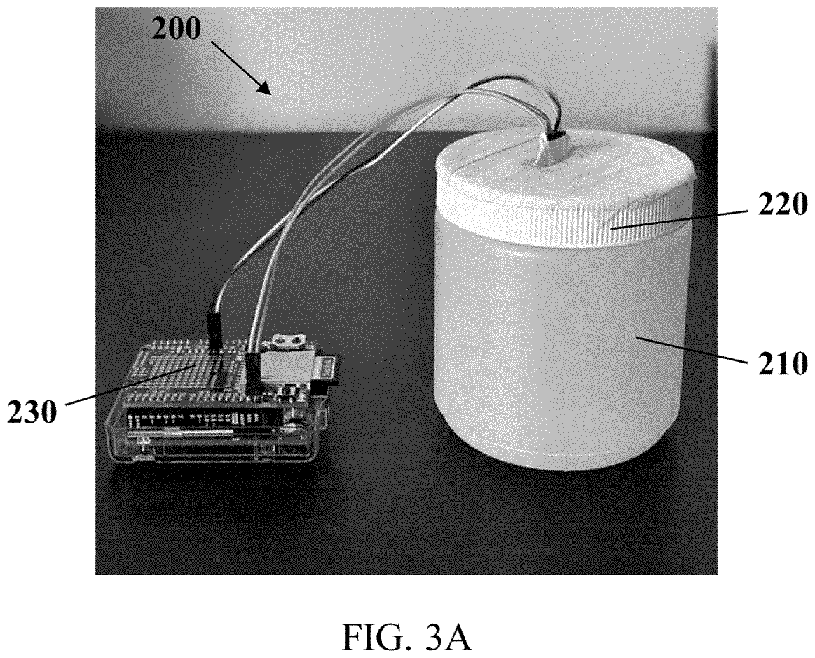

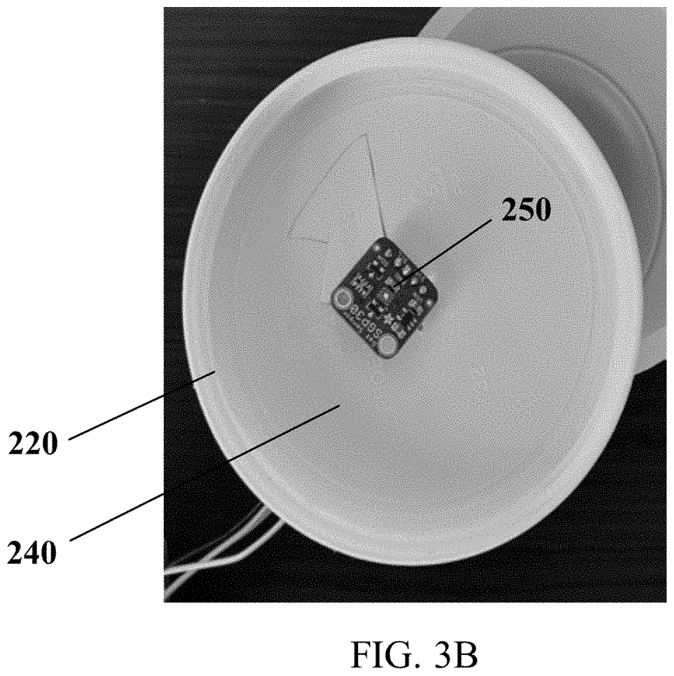

FIGS. 3A and 3B show alternative views of another embodiment of a container 200 as described herein. FIG. 3A shows a container 200 comprising a body 210, a lid 220, a sensing unit (not sown), and a processing unit 230. The lid 220 is coupled to the body 210 of the container 200. FIG. 3B shows an alternative view of the lid 220 of the container 200 shown in FIG. 3A. The lid 220 of FIG. 3B has an interior surface 240, and a sensing unit 250 affixed to the interior surface 240 of the lid 220. The sensing unit 250 is affixed to the interior surface 240 of the lid 220 such that when the lid 220 and the body 210 are coupled, the sensing unit 250 is enclosed within the interior compartment of the body 210 of the container 200.

In an embodiment, a method for monitoring the degradation of a biological tissue may comprise obtaining a container having a body, a lid, a sensing unit, and a processing unit, as described herein. The container may have any or all of the features and configurations described herein.

In some embodiments, the method may further comprise placing a biological tissue in the interior compartment of the body of the container. The biological tissue may comprise, for example, one or more excised tissues, one or more biopsy samples, one or more tissue samples, one or more cells, one or more types of tissue, and the like.

In certain embodiments, the method may further comprise coupling the lid to the body, as described herein. In some embodiments, the method may further comprise contacting the lid and the body, as described herein. As described herein, the processing unit of the container may be configured to be activated and/or deactivated when the configuration of the container is changed, such that the step of coupling or contacting the lid and the body may also comprise activating at least one of the processing unit and the sensing unit. In other embodiments, the method may further comprise uncoupling the lid and the body, and the step of uncoupling the lid and the body may also comprise deactivating at least one of the processing unit and the sensing unit. In some embodiments, at least one of the sensing unit and the processing unit may remain activated as long as the lid and the body of the container remain coupled. In certain embodiments, at least one of the sensing unit and the processing unit may remain activated for a predetermined period of time, or may be deactivated after a predetermined period of time.

In some embodiments, the method may further comprise activating at least one of the sensing unit and the processing unit. In certain embodiments, activating at least one of the sensing unit and the processing unit may thereby execute the instructions stored within the processor-readable storage medium of the processing unit, and thus may determine the status based on the data about the one or more environmental conditions obtained by the sensing unit.

In some embodiments, the method may further comprise observing the indicator, and thereby monitoring the degradation of the biological tissue. In certain embodiments, observing the indicator may be accomplished without uncoupling the lid of the container from the body of the container. As described herein, the indicator may comprise a display. In certain embodiments, the indicator may comprise at least one light emitting diode (LED). In some embodiments, the indicator may be configured to display one or more indications that correspond to the status determined by the processor. In an embodiment, the one or more indications may include, for example, a first color when the status is “viable,” a second color when the status is “mildly degraded,” a third color when the status is “fully degraded,” or any combination thereof. In an embodiment, each color may be any conventional color; for example, the first color may be green, the second color may be yellow, the third color may be red, or any other color scheme. In some embodiments, the step of observing the indicator may comprise observing or viewing the color of the indicator. In an embodiment, the color of the indicator may thereby communicate the viability or degradation of the biological tissue in the interior compartment of the container to an observer.

In certain embodiments, the method may further comprise downloading the data about the environmental condition, or a subset thereof. In certain embodiments, the method may further comprise analyzing the data about the environmental condition, or a subset thereof. The steps of downloading and analyzing the data may each independently be performed while the container, including the processing unit and the sensing unit, are in use, or after their use is complete.

The following are non-limiting embodiments of the present disclosure.

Embodiment 1 is a container comprising: a body having an interior compartment; a lid having an interior surface and a sensing unit affixed to the interior surface; wherein the lid is configured to be coupled to the body, thereby enclosing the sensing unit within the interior compartment of the body, and wherein the sensing unit is configured to obtain data about an environmental condition; and a processing unit in electrical communication with the sensing unit, the processing unit comprising: a processor, a non-transitory, processor-readable storage medium in electrical communication with the processor, a power source in electrical communication with the processor and the processor-readable storage medium, and an indicator in electrical communication with the processor, the processor-readable storage medium, and the power source, wherein the processor-readable storage medium contains instructions that, when executed, cause the processor to: process and store the data about the environmental condition, and determine a status based on the data; and wherein the indicator is configured to display an indication corresponding to the status.

Embodiment 2 is the container of embodiment 1, wherein the interior compartment of the body is configured to house one or more biological tissues.

Embodiment 3 is the container of embodiment 1, wherein the sensing unit comprises a device selected from the group consisting of an electrochemical sensor, a gas sensor, a metal oxide sensor, a gas chromatography apparatus, a mass spectrometry apparatus, a photo-ionization detector, and combinations thereof.

Embodiment 4 is the container of embodiment 1, wherein the sensing unit is configured to detect volatile organic compounds (VOCs).

Embodiment 5 is the container of embodiment 1, wherein the sensing unit comprises a metal oxide sensor configured to detect volatile organic compounds (VOCs).

Embodiment 6 is the container of embodiment 1, wherein the environmental condition is selected from the group consisting of a temperature, a presence of volatile organic compounds (VOCs), a humidity level, an oxygen saturation level, a pH, and combinations thereof.

Embodiment 7 is the container of embodiment 1, wherein the environmental condition comprises a temperature from about 18 degrees Celsius to about 25 degrees Celsius.

Embodiment 8 is the container of embodiment 1, wherein the environmental condition comprises a presence of volatile organic compounds (VOCs) from about 1 ppb to about 60,000 ppb.

Embodiment 9 is the container of embodiment 1, wherein the processor-readable storage medium is selected from the group consisting of a secure digital (SD) card, a receiver wirelessly coupled to an external server, and combinations thereof.

Embodiment 10 is the container of embodiment 1, wherein the indicator comprises at least one light emitting diode (LED).

Embodiment 11 is the container of embodiment 10, wherein the indication corresponding to the status is a color, and wherein the LED is configured to display the color.

Embodiment 12 is the container of embodiment 10, wherein the status is selected from the group consisting of viable, mildly degraded, fully degraded, or combinations thereof.

Embodiment 13 is the container of embodiment 12, wherein the indication is at least one of a first color when the status is viable, a second color when the status is mildly degraded, and a third color when the status is fully degraded.

Embodiment 14 is the container of embodiment 1, wherein the power source comprises a battery.

Embodiment 15 is the container of embodiment 1, wherein the processing unit is configured to be activated when the lid is coupled to the body.

Embodiment 16 is the container of embodiment 1, wherein the processing unit is further configured to record a time of activation, a time of use, and combinations thereof.

Embodiment 17 is the container of embodiment 1, wherein the processing unit is configured to determine the status by comparing the data to a predetermined threshold.

Embodiment 18 is the container of embodiment 1, wherein the container is a portable sterile biopsy container.

Embodiment 19 is a method for monitoring degradation of a biological tissue, the method comprising: obtaining a container comprising: a body having an interior compartment; a lid having an interior surface and a sensing unit affixed to the interior surface; wherein the lid is configured to be coupled to the body, thereby enclosing the sensing unit within the interior compartment of the body, and wherein the sensing unit is configured to obtain data about an environmental condition; and a processing unit in electrical communication with the sensing unit, the processing unit comprising: a processor, a non-transitory, processor-readable storage medium in electrical communication with the processor, a power source in electrical communication with the processor and the processor-readable storage medium, and an indicator in electrical communication with the processor, the processor-readable storage medium, and the power source, wherein the processor-readable storage medium contains instructions that, when executed, cause the processor to: process and store the data about the environmental condition, and determine a status based on the data; and wherein the indicator is configured to display an indication corresponding to the status; and placing the biological tissue in the interior compartment of the body; coupling the lid to the body; activating the sensing unit and the processing unit, thereby executing the instructions and determining the status; and observing the indicator, thereby monitoring the degradation of the biological tissue.

Embodiment 20 is the method of embodiment 19, further comprising: downloading the data about the environmental condition; and analyzing the data about the environmental condition.

Embodiment 21 is the method of embodiment 19, wherein the sensing unit comprises a device selected from the group consisting of an electrochemical sensor, a gas sensor, a metal oxide sensor, a gas chromatography apparatus, a mass spectrometry apparatus, a photo-ionization detector, and combinations thereof.

Embodiment 22 is the method of embodiment 19, wherein the sensing unit is configured to detect volatile organic compounds (VOCs).

Embodiment 23 is the method of embodiment 19, wherein the sensing unit comprises a metal oxide sensor configured to detect volatile organic compounds (VOCs), and wherein the status is based on a presence of VOCs.

Embodiment 24 is the method of embodiment 19, wherein the environmental condition is selected from the group consisting of a temperature, a presence of volatile organic compounds (VOCs), a humidity level, an oxygen saturation level, a pH, and combinations thereof.

Embodiment 25 is the method of embodiment 19, wherein the environmental condition comprises a temperature from about 18 degrees Celsius to about 25 degrees Celsius.

Embodiment 26 is the method of embodiment 19, wherein the environmental condition comprises a presence of volatile organic compounds (VOCs) from about 1 ppb to about 60,000 ppb.

Embodiment 27 is the method of embodiment 19, wherein the processor-readable storage medium is selected from the group consisting of a secure digital (SD) card, a receiver wirelessly coupled to an external server, and combinations thereof.

Embodiment 28 is the method of embodiment 19, wherein the indicator comprises at least one light emitting diode (LED).

Embodiment 29 is the method of embodiment 28, wherein the indication corresponding to the status is a color, wherein the LED is configured to display the color, and wherein observing the indicator comprises viewing the color.

Embodiment 30 is the method of embodiment 28, wherein the status is selected from the group consisting of viable, mildly degraded, fully degraded, or combinations thereof.

Embodiment 31 is the method of embodiment 30, wherein the indication is at least one of a first color when the status is viable, a second color when the status is mildly degraded, and a third color when the status is fully degraded.

Embodiment 32 is the method of embodiment 19, wherein the power source comprises a battery.

Embodiment 33 is the method of embodiment 19, wherein coupling the lid to the body activates the sensing unit and the processing unit.

Embodiment 34 is the method of embodiment 19, wherein the processing unit is further configured to record a time of activation, a time of use, and combinations thereof.

Embodiment 35 is the method of embodiment 19, wherein the processing unit is configured to determine the status by comparing the data to a predetermined threshold.

Embodiment 36 is the method of embodiment 19, wherein the container is a portable sterile biopsy container.

### EXAMPLES

### Example 1: Monitoring Emission of VOCs Over Time

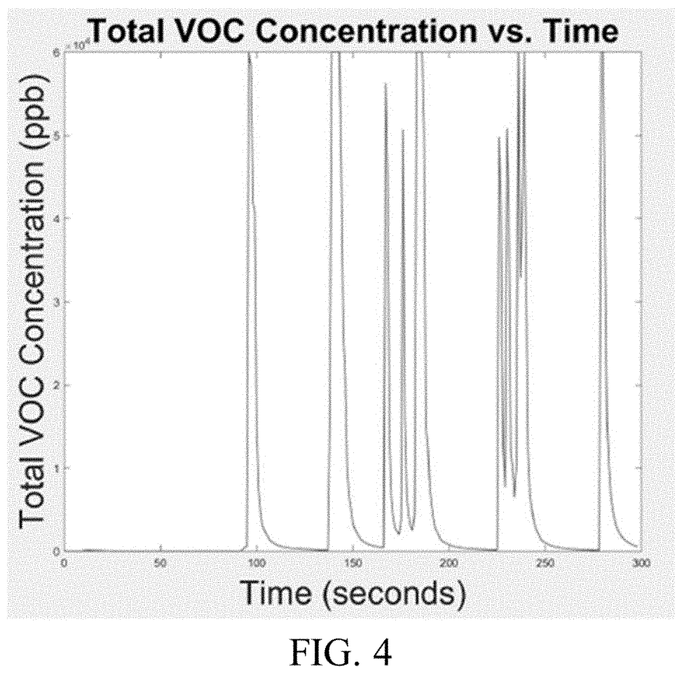

A container having a body, a lid, a sensing unit, and a processing unit, as described herein, was produced. To illustrate the sensing unit's ability to detect VOC emission, the sensing unit was connected to a computer and data acquisition was initiated. A cotton swab was soaked in ethyl alcohol, and then moved in close proximity to, and then away from, the sensing unit several times. FIG. 4 is a graph showing the total concentration of VOCs (in ppb) detected during the period of data acquisition, with peaks corresponding to periods when the soaked cotton swab was moved in close proximity to the sensing unit. The acquired data demonstrated that the sensing unit was able to detect VOC concentrations from about 1 ppb to about 60,000 ppb.

### Example 2: Monitoring Relative Emission of VOCs from Different Samples Over Time

Six containers, each container having a body, a lid, a sensing unit, and a processing unit, as described herein, were produced. Six dilutions of ethyl alcohol were prepared as shown in Table 2 below:

TABLE 2

| Sample No. | Ethyl alcohol (mL) | Filtered water (mL) |
|---|---|---|
| 1 | 5 | 240 |
| 2 | 10 | 240 |
| 3 | 15 | 240 |
| 4 | 20 | 240 |
| 5 | 25 | 240 |
| 6 | 0 | 240 |

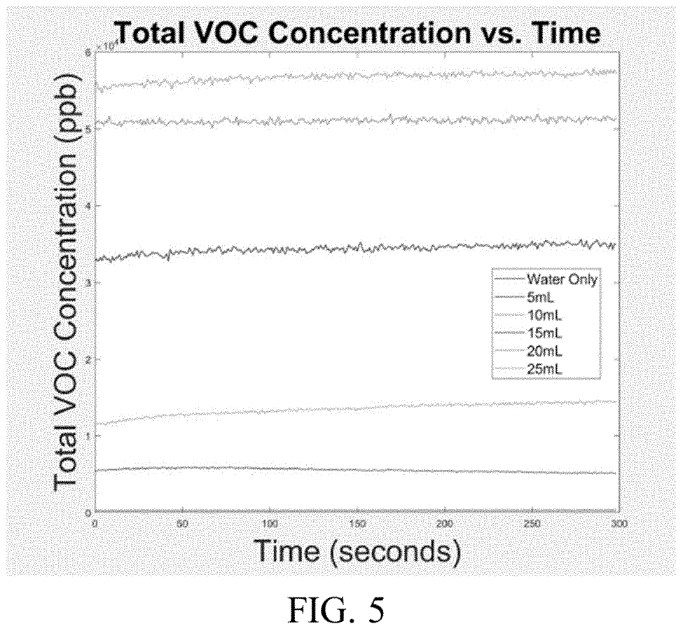

The dilutions with higher concentrations of ethyl alcohol were expected to demonstrate proportionally higher levels of VOC emissions. Each dilution was placed in a separate container, and data was collected and recorded from each container as described herein for a total of 300 seconds. The data was analyzed to determine the total VOC concentration as a function of time. FIG. 5 shows the total concentration of volatile organic compounds (VOCs, in ppb) as a function of time (in seconds) resulting from this experiment.

### Example 3: VOCs Increase Over Time Under Pro-Degradation Conditions

### Methods

Three Sprague Dawley male rats aged 90-120 days were utilized to monitor VOC emission under tissue degradation conditions. The animals were euthanized with CO2 and their livers resected and separated into 6 samples of approximately 20 mg size. Samples were placed in designated sample vials and were allowed to incubate at room temperature until a designated time point at which the samples were transferred to dry ice. VOC analysis was conducted via GC-MS. Solid phase microextraction (SPME) was used to extract the samples from the tissue headspace.

The instrumentation conditions included an inlet temperature of 250° C., a transfer line temperature of 240° C., a column helium (the carrier gas) flow rate of 1.4 mL/min, and a J&W DB-5 column of dimensions 30 m×250 μm×0.50 μm. The oven within the GC unit began at 40° C. for 3 minutes, then increased at a rate of 10° C./min to 300° C., and then held at that temperature for 3 minutes. The MS source had a temperature of 230° C. and the MS quadrupoles had a temperature of 150° C.

The samples were first brought to the agitator, which created motion within the sample to release the VOCs. The agitator was not heated, and thus worked at room temperature. The SPME fiber was then inserted into the sample vial, and the extraction time was 30 minutes per sample. After this time, the fiber was placed at the GC inlet. Here, the desorption time was 1 minute per sample. Then, the fiber was exposed to helium at 300° C. for 1 minute to ensure all compounds had been released from the fiber. The compounds were then separated within the GC column.

Once the samples were run through the whole GC-MS process, the gathered data was analyzed using the Agile 2 algorithm. Peaks that were at least 0.1% of the highest peak were included, and the NIST17 database was used for matching the experimental compounds to the library. Peaks were named if the match score was greater than 50. The final spreadsheet contained the peak name, compound molecular formula, peak area, match score, and retention time. For analysis, compounds containing Si were removed because these occurred as a result of the fiber and/or cap septa used for testing, not from the sample. Then, the remaining peak areas were added to find the total peak area. Finally, the percent of the peak area was found for each compound with a match score greater than 50 within each sample

### Results

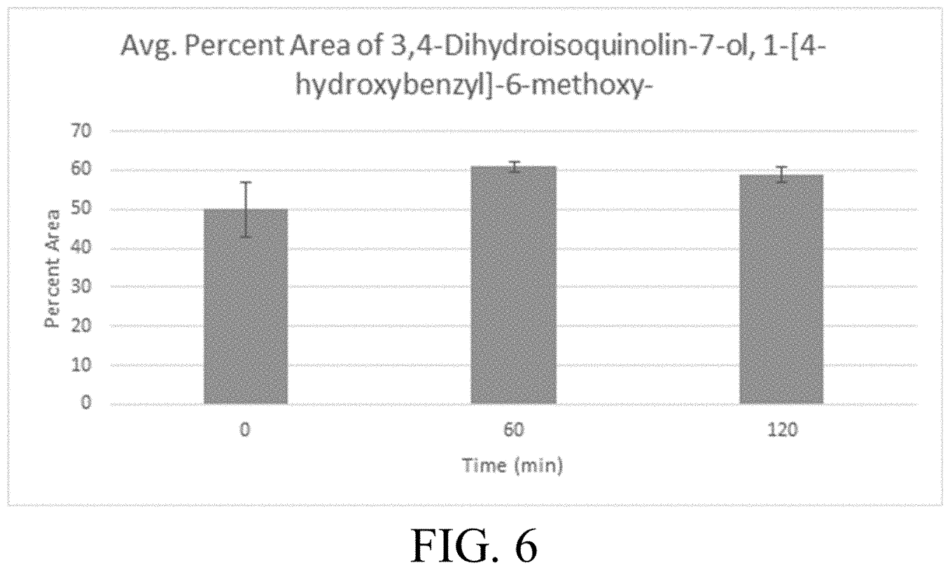

There were a total of 1,269 compounds identified within the nine experimental samples. Only 141 of these compounds had a match score greater than 50, so there were 1,128 compounds negated from the following results. Of the 141 named compounds, there were 93 unique compounds. VOC compounds 3,4-Dihydroisoquinolin-7-ol, 1-[4-hydroxybenzyl]-6-methoxy-, 3,4-Dimethoxycinnamic acid, and 1,2,4-Benzenetricarboxylic acid, 1,2-dimethyl ester were found to comprise a majority of these samples. FIG. 6 illustrates the relative concentration of 3,4-Dihydroisoquinolin-7-ol, 1-[4-hydroxybenzyl]-6-methoxy- at specific room temp incubation times. The data show that as tissue samples are allowed to incubate under pro-degradation conditions an increase in 3,4-Dihydroisoquinolin-7-ol, 1-[4-hydroxybenzyl]-6-methoxy- is observed.

### Example 4: Detection of VOC Content—Using GC/MS and Tissue Status Monitoring Biopsy Container

As detailed in Example 3, the content of 3,4-Dihydroisoquinolin-7-ol, 1-[4-hydroxybenzyl]-6-methoxy- was increased in the headspace of tissue samples under pro-degradation conditions as analyzed via GC/MS. The GC/MS analysis and the function of an embodiment of a container as described herein will be compared.

### Methods

The method for the procedure will be largely the same as outlined in Example 3 above. Samples will be collected and allowed to incubate under pro-degradation conditions. Samples placed in the embodiment of the container will be constantly assessed via readout of the indicator throughout the duration of the experiment. As in Example 3, the headspace of samples designated for GC/MS analysis will be collected at specific time points and analyzed. Direct comparison of tissue status via the container and the VOC content via GC/MS will be performed.

The sensitivity of the container will allow for detection of VOC content in the ppb range. Thus, the container will detect changes in total VOC content in the tissue sample over time. GC/MS analysis will identify the exact species of VOCs being emitted from the tissue samples as the sample quality deteriorates.

While the present disclosure has been illustrated by the description of exemplary embodiments thereof, and while the embodiments have been described in certain detail, the Applicant does not intend to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

In the above detailed description, reference is made to the accompanying drawings, which form a part hereof In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A container comprising:
a body having an interior compartment;
a lid having an interior surface and a sensing unit affixed to the interior surface;
wherein the lid is configured to be coupled to the body, thereby enclosing the sensing unit within the interior compartment of the body, and
wherein the sensing unit is configured to obtain data about an environmental condition; and
a processing unit in electrical communication with the sensing unit, the processing unit comprising:
a processor,
a non-transitory, processor-readable storage medium in electrical communication with the processor,
a power source in electrical communication with the processor and the processor-readable storage medium, and
an indicator in electrical communication with the processor, the processor-readable storage medium, and the power source,
wherein the processor-readable storage medium contains instructions that, when executed, cause the processor to:
process and store the data about the environmental condition, and determine a status based on the data; and
wherein the indicator is configured to display an indication corresponding to the status.

2. The container of claim 1, wherein the interior compartment of the body is configured to house one or more biological tissues.

3. The container of claim 1, wherein the sensing unit comprises a device selected from the group consisting of an electrochemical sensor, a gas sensor, a metal oxide sensor, a gas chromatography apparatus, a mass spectrometry apparatus, a photo-ionization detector, and combinations thereof.

4. The container of claim 1, wherein the environmental condition is selected from the group consisting of a temperature, a presence of volatile organic compounds (VOCs), a humidity level, an oxygen saturation level, a pH, and combinations thereof.

5. The container of claim 1, wherein the sensing unit is configured to detect volatile organic compounds (VOCs) in an amount from about 1 ppb to about 60,000 ppb.

6. The container of claim 1, wherein the sensing unit comprises a metal oxide sensor configured to detect volatile organic compounds (VOCs).

7. The container of claim 1, wherein the processor-readable storage medium is selected from the group consisting of a secure digital (SD) card, a receiver wirelessly coupled to an external server, and combinations thereof.

8. The container of claim 1, wherein the indicator comprises at least one light emitting diode (LED).

9. The container of claim 8, wherein the status is selected from the group consisting of viable, mildly degraded, fully degraded, or combinations thereof, wherein the indication corresponding to the status is a color, and wherein the LED is configured to display the color.

10. The container of claim 9, wherein the indication is at least one of a first color when the status is viable, a second color when the status is mildly degraded, and a third color when the status is fully degraded.

11. The container of claim 1, wherein the processing unit is configured to be activated when the lid is coupled to the body.

12. The container of claim 1, wherein the processing unit is further configured to record a time of activation, a time of use, and combinations thereof.

13. The container of claim 1, wherein the processing unit is configured to determine the status by comparing the data to a predetermined threshold.

14. A container comprising:
a body having an interior compartment; configured to house one or more biological tissues;
a lid having an interior surface and a sensing unit affixed to the interior surface;
wherein the lid is configured to be coupled to the body, thereby enclosing the sensing unit within the interior compartment of the body, and
wherein the sensing unit comprises a metal oxide sensor configured to detect volatile organic compounds (VOCs) in an amount from about 1 ppb to about 60,000 ppb; and
a processing unit in electrical communication with the sensing unit, the processing unit comprising:
a processor,
a non-transitory, processor-readable storage medium in electrical communication with the processor,
wherein the processor-readable storage medium is selected from the group consisting of a secure digital (SD) card, a receiver wirelessly coupled to an external server, and combinations thereof;
a power source in electrical communication with the processor and the processor-readable storage medium, wherein the power source comprises a battery; and
an indicator in electrical communication with the processor, the processor-readable storage medium, and the power source, wherein the indicator comprises at least one light emitting diode (LED);
wherein the processor-readable storage medium contains instructions that, when executed, cause the processor to:
process and store the data about the environmental condition, and determine a status based on the data; and
wherein the indicator is configured to display an indication corresponding to the status, wherein the status is selected from the group consisting of viable, mildly degraded, fully degraded, or combinations thereof,
wherein the indication is at least one of a first color when the status is viable, a second color when the status is mildly degraded, and a third color when the status is fully degraded;
wherein the LED is configured to display the color; and
wherein the processing unit is configured to be activated when the lid is coupled to the body.

15. A method for monitoring degradation of a biological tissue, the method comprising:
obtaining a container comprising:
a body having an interior compartment;
a lid having an interior surface and a sensing unit affixed to the interior surface;
wherein the lid is configured to be coupled to the body, thereby enclosing the sensing unit within the interior compartment of the body, and
wherein the sensing unit is configured to obtain data about an environmental condition; and
a processing unit in electrical communication with the sensing unit, the processing unit comprising:
a processor,
a non-transitory, processor-readable storage medium in electrical communication with the processor,
a power source in electrical communication with the processor and the processor-readable storage medium, and
an indicator in electrical communication with the processor, the processor-readable storage medium, and the power source,
wherein the processor-readable storage medium contains instructions that, when executed, cause the processor to:
process and store the data about the environmental condition, and
determine a status based on the data; and
wherein the indicator is configured to display an indication corresponding to the status; and
placing the biological tissue in the interior compartment of the body;
coupling the lid to the body;
activating the sensing unit and the processing unit, thereby executing the instructions and determining the status; and
observing the indicator, thereby monitoring the degradation of the biological tissue.

16. The method of claim 15, further comprising:
downloading the data about the environmental condition; and
analyzing the data about the environmental condition.

17. The method of claim 15, wherein the sensing unit comprises a device selected from the group consisting of an electrochemical sensor, a gas sensor, a metal oxide sensor, a gas chromatography apparatus, a mass spectrometry apparatus, a photo-ionization detector, and combinations thereof.

18. The method of claim 15, wherein the processing unit is further configured to record a time of activation, a time of use, and combinations thereof.

19. The method of claim 15, wherein the processing unit is configured to determine the status by comparing the data to a predetermined threshold.

20. The method of claim 15, wherein:
the interior compartment of the body is configured to house one or more biological tissues;
the sensing unit comprises a metal oxide sensor configured to detect volatile organic compounds (VOCs) in an amount from about 1 ppb to about 60,000 ppb;
the processor-readable storage medium is selected from the group consisting of a secure digital (SD) card, a receiver wirelessly coupled to an external server, and combinations thereof;
the power source comprises a battery;
the indicator comprises at least one light emitting diode (LED);
the status is selected from the group consisting of viable, mildly degraded, fully degraded, or combinations thereof,
the indication is at least one of a first color when the status is viable, a second color when the status is mildly degraded, and a third color when the status is fully degraded;
the LED is configured to display the color; and
the processing unit is configured to be activated when the lid is coupled to the body.
